(19) United States
(12) Patent Application Publication
(10) Pub. No.: US 2008/0249407 A1
(43) Pub. Date: Oct. 9, 2008

(54) User Interface System and Method for Creating, Organizing and Setting-Up Ultrasound Imaging Protocols

(75) Inventors: Steven J. Hill, Bothell, WA (US); Cedric Chenal, Kirkland, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., Eindhoven (NL)

(21) Appl. No.: 12/088,100

(22) PCT Filed: Sep. 26, 2006
(86) PCT No.: PCT/IB06/53503

§ 371 Date: Mar. 26, 2008

(30) Foreign Application Priority Data
Sep. 30, 2005 (US) 60722293

(51) Int. Cl. A61B 8/00

(52) U.S. Cl. 600/437

(57) ABSTRACT

An ultrasound imaging system includes a display for displaying an ultrasound image and an analysis package operatively connected to the image display. The analysis package provides a user the ability to manage an ultrasound imaging protocol. The analysis package facilitates the management of the imaging protocol by displaying for the sonographer an interface which allows for editing, sorting and organizing protocol views using representative images.

This invention relates to medical ultrasound imaging systems and, more particularly, to a user interface in an ultrasound imaging system that allows for creating, organizing and setting-up ultrasound imaging protocols.

In the past, general purpose ultrasound imaging systems were used to provide images of all anatomical features that could be imaged using ultrasound. But as ultrasound diagnosis has become more sophisticated and the technology more refined, ultrasound imaging systems have become more specialized and configured for imaging specific anatomy during certain specific types of examinations such as obstetrics, cardiology, vascular and radiology. In the recent past the practice of ultrasound diagnosis has become more standardized, with specific image acquisition protocols being designed for patients with specific symptoms or characteristics. For example, a general abdominal exam protocol may call for the acquisition of particular views of the liver, kidneys, gall bladder and pancreas. A general vascular exam may call for the acquisition of particular views of the carotid artery and vasculature of the limbs of the body. Ultrasound imaging system manufacturers have followed this trend by providing their systems with pre-programmed exam protocols to guide the sonographer through the gathering of these specific image sequences. These pre-programmed exam protocols also cause ultrasound imaging systems to produce reports automatically tailored to the specified information. Such pre-programmed protocols have improved the efficiency of conducting ultrasound exams.

Pre-programmed protocols, particularly those for a general survey exam, are generally designed to step the sonographer through a series of views, measurements, and calculations in particular regions of the body to determine whether the imaged anatomy is normal or exhibits suspect characteristics. In addition to these pre-programmed protocols, more sophisticated ultrasound imaging systems typically allow the sonographer to design custom protocols, which include custom ultrasound image acquisition ordering, system setup, measurements and calculations not provided by a default protocol in the ultrasound imaging system. This valuable feature frees the sonographer from the restriction of using only the protocols provided with the ultrasound imaging system and variations thereof, and enables sonographers and researchers to develop their own new and more efficient protocols and system configurations.

Although conventional ultrasound imaging systems allow custom protocols to be created, limitations in their user interfaces make it cumbersome and non-intuitive to develop and implement custom protocols, or to re-configure and organize existing protocols. In such prior art systems, the user interface presented to the sonographer for creating or editing custom protocols is typically monolithic and features a single screen with many data fields. Any particular field may or may not be required for completing a particular operation. Which fields are, in fact, required is not immediately obvious. Such an interface is not intuitive and imposes on the sonographer the burden of learning how to manipulate the interface to create or edit a custom protocol. Because of these limitations, labs and clinics are often unable to custom-configure their ultrasound imaging system protocols without the time consuming and expensive assistance of a service representative.

There is therefore a need for an ultrasound imaging system that provides the sonographer with a more streamlined and intuitive method and interface for creating, organizing and setting-up custom protocols.

The invention is directed to a medical ultrasound imaging system and method for creating and managing custom ultrasound imaging protocols. A sonographer may use such equipment to perform ultrasound examinations such as, for example, a cardiac examination. The ultrasound equipment is configured by a protocol which also guides the sonographer through each view of the examination and dictates the types of images and measurements the sonographer should perform during the examination. These images and measurements are then used to derive various calculations useful for clinical and diagnostic purposes. The ultrasound imaging system and method provides a user interface for creating and managing new custom protocols beyond those that are provided by the manufacturer. The system and method provides a display of images representative of the views to be acquired during the execution of the protocol. These images can be dragged and dropped into a new or pre-existing protocol to add new views to the protocol or place the existing images of the protocol in a different order. Similarly, exam stages within the protocol, and the views within each stage, can be re-ordered by dragging and dropping the representative images in desired locations. A custom protocol created in this manner can be saved in the system for later recall. Display of exam views via icon-like representative images increases sonographer efficiency by providing a streamlined and intuitive method and interface for creating and editing custom protocols.

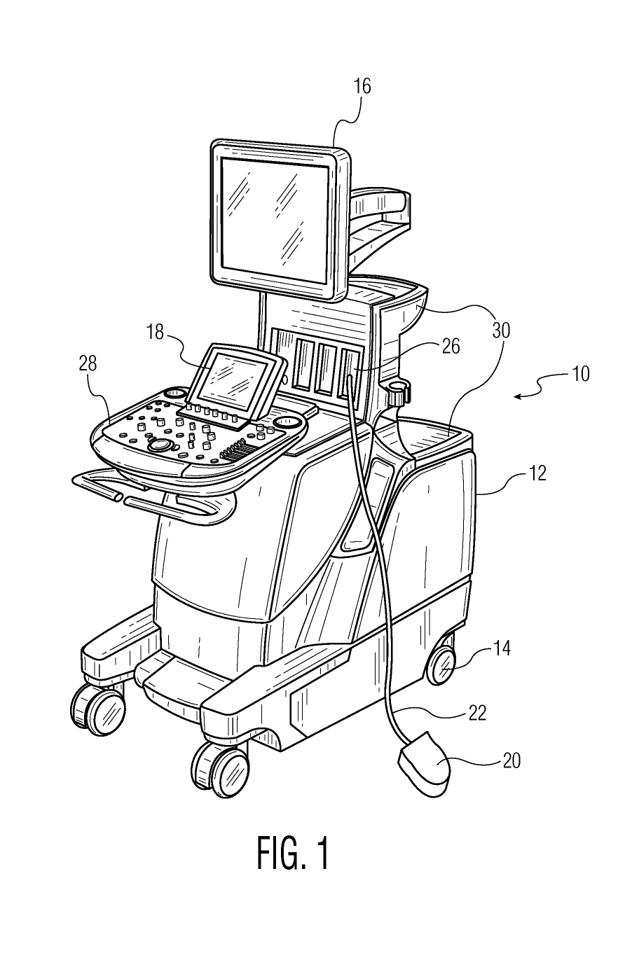

FIG. 1 is an isometric view of an ultrasound imaging system according to one example of the invention.

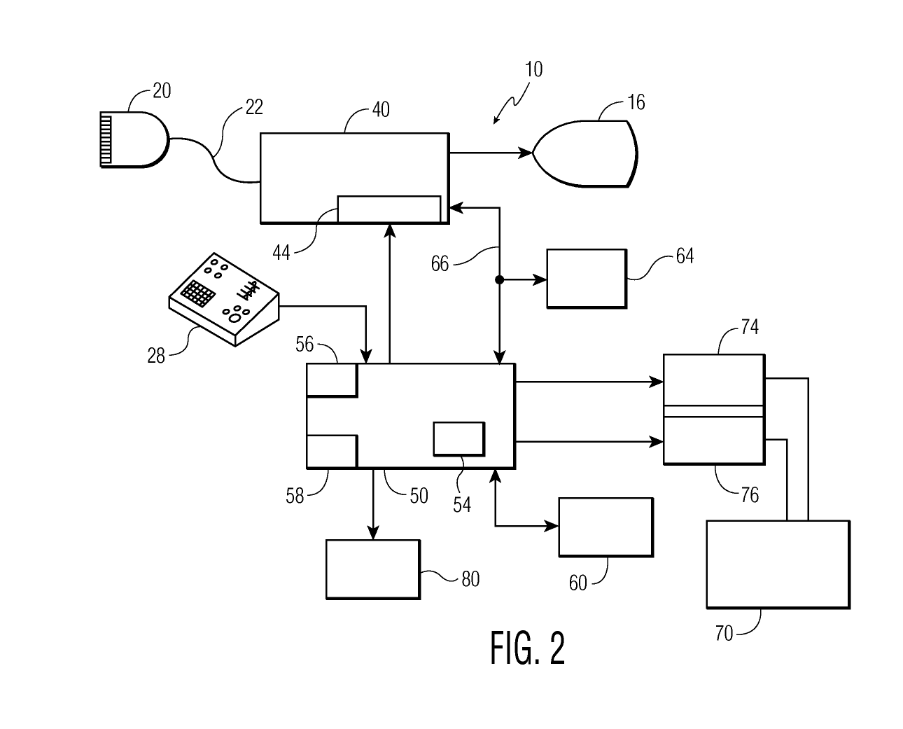

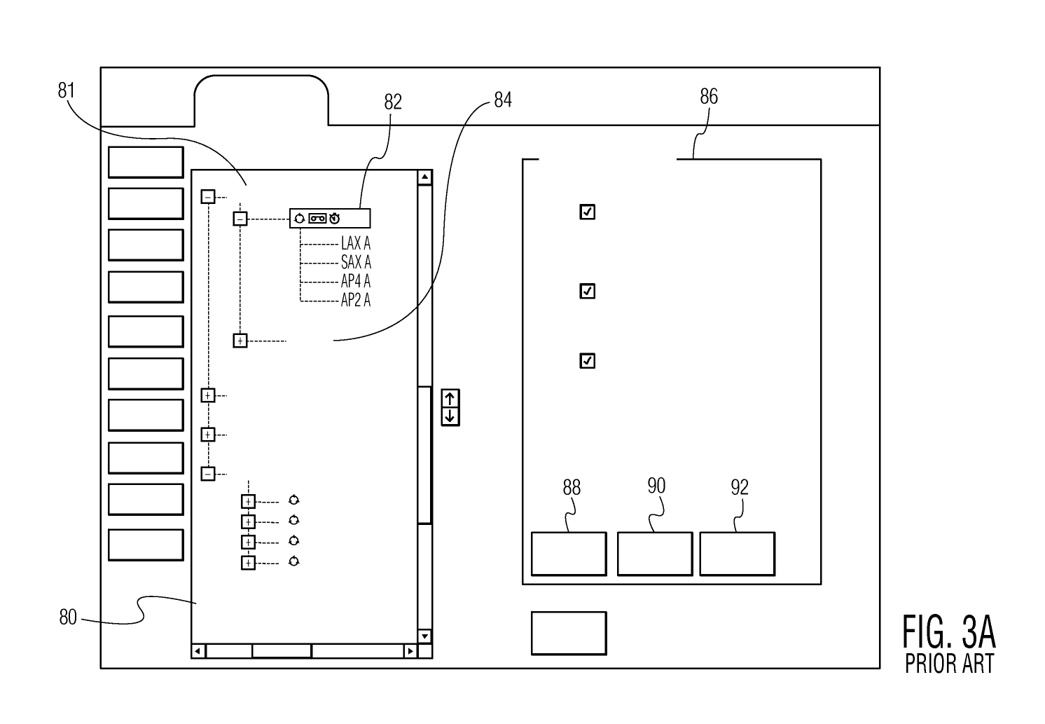

FIG. 2 is a block diagram of the electrical components used in the ultrasound imaging system of FIG. 1.

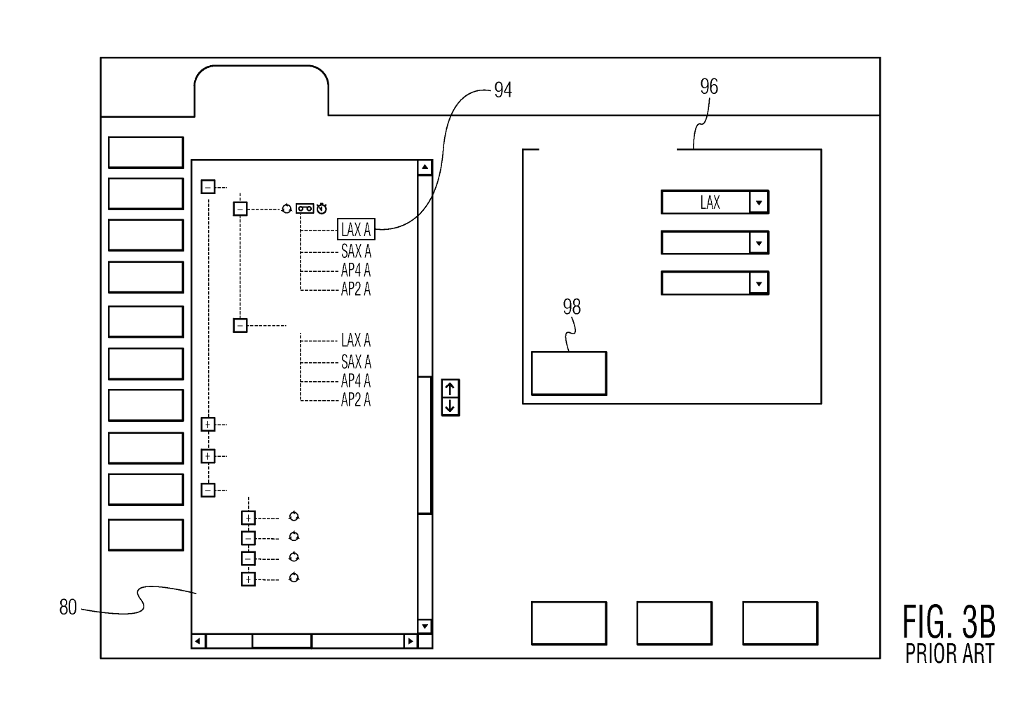

FIGS. 3a and 3b illustrate examples of a prior art user interface used in conjunction with managing ultrasound imaging protocols.

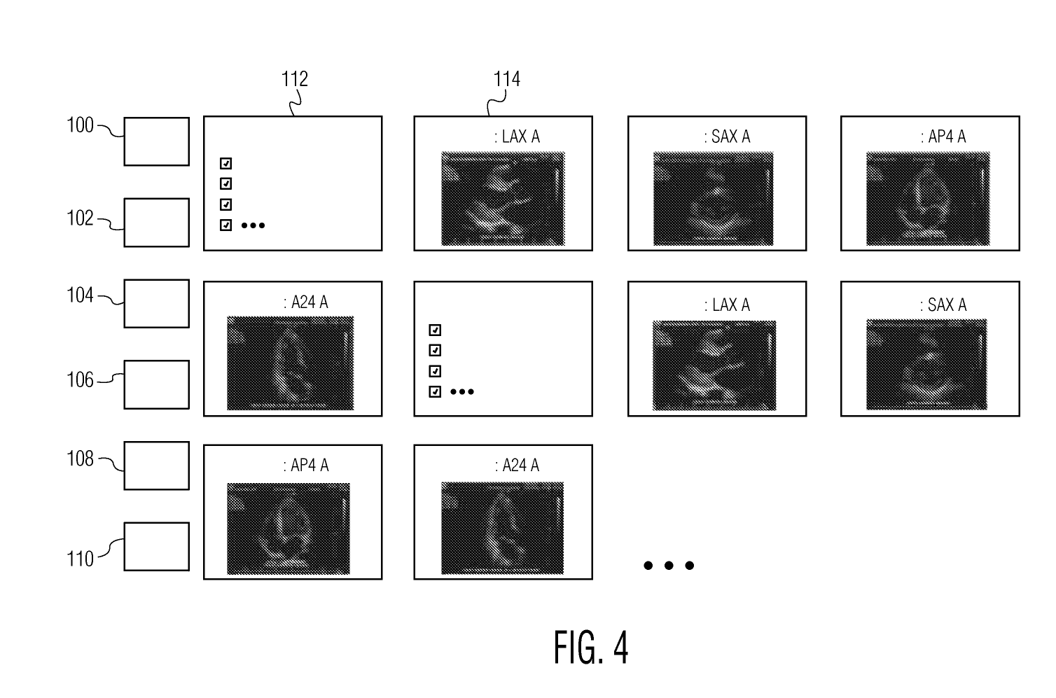

FIG. 4 illustrates a user interface according to one example of the present invention.

An ultrasound imaging system 10 according to one example of the invention is illustrated FIG. 1. The system 10 includes a chassis 12 containing most of the electronic circuitry for the system 10. The chassis 12 is mounted on a cart 14, and a display 16 is mounted on the chassis 12. An imaging probe 20 is connected through a cable 22 to one of three connectors 26 on the chassis 12. The chassis 12 includes a keyboard and controls, generally indicated by reference numeral 28, for allowing a sonographer to operate the ultrasound imaging system 10 and enter information about the patient or the type of examination that is being conducted. At the back of the control panel 28 is a touchscreen display 18 on which programmable softkeys are displayed for supplementing the keyboard and controls 28 in controlling the operation of the system 10.

In operation, the imaging probe 20 is placed against the skin of a patient (not shown) and held stationary to acquire an image of blood or tissues in a volumetric region beneath the skin. A planar or volumetric image is presented on the display 16, and the image may be recorded by a recorder (not shown) placed on one of the two accessory shelves 30. The system 10 may also record or print a report containing text and images. Data corresponding to the image may also be downloaded through a suitable data link, such as the Internet or a local area network. In addition to using the probe 20 to show an image on the display, the ultrasound imaging system may also provide other types of information useful for a diagnosis, and it may accept other types of probes (not shown) to provide other types of images.

The electrical components of the ultrasound imaging system 10 are illustrated in FIG. 2. As mentioned above, the ultrasound imaging probe 20 is coupled by the cable 22 to one of the connectors 26, which are connected to an ultrasound signal path 40 of conventional design. As is well-known in the art, the ultrasound signal path 40 includes a transmitter (not shown) coupling electrical signals to the probe 20, an acquisition unit (not shown) that receives electrical signals from the probe 20 corresponding to ultrasound echoes, a signal processing unit (not shown) that processes the signals from the acquisition unit to perform a variety of functions such as isolating returns from specific depths or isolating returns from blood flowing through vessels, and a scan converter (not shown) that converts the signals from the signal processing unit so that they are suitable for use by the display 16. The processing unit in this example is capable of processing both B mode (structural) and Doppler signals for the production of various B mode and Doppler images, including spectral Doppler images. The ultrasound signal path 40 also includes a control module 44 that interfaces with a processing unit 50 to control the operation of the above-described units. The ultrasound signal path 40 may, of course, contain components in addition to those described above, and, in suitable instances, some of the components described above may be omitted.

The processing unit 50 contains a number of components, including a central processor unit (“CPU”) 54, random access memory (“RAM”) 56, and read only memory (“ROM”) 58, to name a few. As is well-known in the art, the ROM 58 stores a program of instructions that are executed by the CPU 54, as well as initialization data for use by the CPU 54. The RAM 56 provides temporary storage of data and instructions for use by the CPU 54. The processing unit 50 interfaces with a mass storage device such as a disk drive 60 for permanent storage of data, such as data corresponding to ultrasound images obtained by the system 10. However, such image data is initially stored in an image storage device 64 that is coupled to a signal path 66 extending between the ultrasound signal path 40 and the processing unit 50. The disk drive 60 also preferably stores protocols which may be called up and initiated to guide the sonographer through various ultrasound exams.

The processing unit 50 also interfaces with the keyboard and controls 28. The keyboard and controls 28 may also be manipulated by the sonographer to cause the ultrasound imaging system 10 to produce automatically generated reports at the conclusion of an examination. The processing unit 50 preferably interfaces with a report printer 80 that prints reports containing text and one or more images. The type of reports provided by the printer 80 depends on the type of ultrasound examination that was conducted by the execution of a specific protocol. Finally, as mentioned above, data corresponding to the image may be downloaded through a suitable data link, such as a network 74 or a modem 76, to a clinical information system 70 or other device.

A typical prior art user interface for managing ultrasound imaging protocols in illustrated in FIGS. 3a and 3b. The left side of both FIGS. 3a and 3b illustrates a protocol tree 80. The protocol tree 80 shows a hierarchical structural rendering of a typical cardiac examination protocol. Such an examination consists of two stages. One stage comprises acquiring ultra sound images and measurements during a pre-exercise or resting state, whereas the second stage usually consists of acquiring the same ultrasound images and measurements immediately post-exercise. As will be understood by one of ordinary skill in the art, these stages are generally referred to as the ‘rest’ and ‘impost’ stages respectively. A protocol stage consists of all the images and measurements acquired during that stage. Each image or measurement is generically called a view.

With reference to FIG. 3a and as has been discussed above, the exercise protocol 81 consists of two stages: stage Rest 82 and stage Impost 84. Stage Rest 82 has been selected and expanded within the hierarchical tree and all of the views of this stage are visible. The views are represented by the textual labels “LAX A,” “SAX A,” “AP4,” and “AP2.” Such labels denote long-axis, short-axis, apical four-chamber and apical two-chamber image views as will be understood by those of ordinary skill in the art. Because stage Rest 82 is selected, the right side of FIG. 3a shows the stage properties dialog 86 that displays the properties of the selected stage and provides the sonographer with buttons for performing management operations on the stage. More specifically, the “Delete Stage” button 88, “Duplicate Stage” button 90 and “Rename Stage” button 92 allow the sonographer to delete, copy and rename the stage.

FIG. 3b differs from FIG. 3a in that the LAX A view 94 is selected instead of stage Rest 82. The left side of FIG. 3b displays the protocol tree [80] whereas the right side shows the view properties dialog 96 because the LAX A view 94 is selected. In addition to displaying the properties of the LAX A view 94, the view properties dialog 96 provides the sonographer with the ability to delete the selected view by pressing the “Delete view” button 98.

This user interface suffers from several shortcomings. The interface is difficult for the layperson to understand, configure and use. In the protocol tree 80, the protocols, and the stages and views within a given protocol, are organized in an unintuitive tree-like format. The properties of each stage and view are not visible until the stage or view is clicked upon and it is not clear to the sonographer what image each view is associated with because each view of the protocol is represented only by an acronym. For example, with reference to FIG. 3b, the first protocol view 94 has been selected and is named “LAX A.” The view properties dialog 96 displays the properties of the LAX A view. None of the properties of any other stage or view are visible in this user interface. In order to view any such properties, a different stage or view must be selected. This makes it difficult to compare various views because the properties for different views cannot be viewed at the same time and the sonographer must go back and forth between views in order to make comparisons. Moreover, several desirable features are missing from this user interface. There is no provision for selecting protocol views from a pre-existing library. There is likewise no provision for copying views between different stages and protocols. The only means of accomplishing this is to copy an entire stage between protocols and remove the unwanted views from the resulting protocol. In sum, there is a distinct lack of configurability in such a system.

FIG. 4 illustrates one example of an improved user interface designed in accordance with the present invention. This example user interface would be used in conjunction with editing an existing protocol. Several buttons 100-110 are presented along the left side of the screen and are used to initiate configuration and management operations on the protocol views and stages.

The “New” button 100 is used to create a new stage or view within a stage. To create a new view within an existing stage, the sonographer would select an existing stage by clicking on its representative image, stage Rest 112 for example, and then clicking the “New” button 100. The system then displays a library of pre-existing views using graphical representations for such views. The graphical representations may be thumbnail images as illustrated in the drawing, or icons representative of particular image views. The sonographer may then either select one of these existing views for inclusion in the protocol, or create and store a new view within the library. The sonographer typically selects one of the existing views by simply dragging the representative image or icon and dropping it into the protocol in the correct place. Creating a new view, on the other hand, is accomplished by clicking on a “New View” button within the library dialog (not shown). This newly created view could then be placed into the stage just like any of the other pre-existing views. Specifically, the user would drag and drop the newly created view into the appropriate place in the protocol. Creating a new stage would progress in a similar manner except that instead of selecting a pre-existing stage, the sonographer would select nothing. Selecting nothing and clicking the “New” button 100 would allow the system to infer that the sonographer wishes to create a new stage and proceed accordingly. Alternatively, after clicking the “New” button 100, the system could prompt the sonographer to specify whether they wish to create a new view or a new stage.

The “Del” button 102 is used to delete an existing stage or view within a protocol. This is achieved by first selecting the graphical representation of the stage or view to be deleted by clicking on its representative image, and then clicking the “Del” button 102. When deleting a stage, all the views within the stage will be deleted as well. Typically, the system will prompt the user to confirm the deletion prior to doing so to ensure that the “Del” button 102 was not clicked accidentally.

The “Cut” button 104 is used to remove a view or stage from its present location for subsequent inclusion of the view or stage in another location. Such an operation would proceed, for example, by first selecting the graphical representation of a view in the protocol by clicking on the graphical representation, and then clicking the “Cut” button 104. The selected view would be removed from its current position, but be held in a temporary location in system memory in a manner that is understood by one of ordinary skill in the art. Next, the sonographer selects a destination location for the view and clicks the “Paste” button 108. The graphical representation of the view is then moved from the temporary location in system memory and placed into the protocol in the location specified. The “Cut” button 104 is most useful for moving protocol views between different protocols. For moving views within the same protocol, and in accordance with the present invention, the sonographer need only drag and drop the graphical representation of any given view from its current location within the protocol to its new position within the protocol. Although discussed in terms of moving a view, the foregoing description applies to stages as well and, as with deleting, cutting and pasting of a stage will result in every view within that stage moving along with the stage itself.

The “Copy” button 106 is used to copy a view or stage for inclusion of that copy in a new location in a protocol. A copy operation would have the sonographer select the graphical representation of a view in the protocol by clicking on that graphical representation, and then clicking the “Copy” button 106. The selected view is copied into a temporary location in system memory. The sonographer then navigates to another location within the same or a different protocol then clicks the “Paste” button 108. The graphical representation of the copied view appears in the specified location and the copy is complete. Again, although discussed in terms of copying a view, the foregoing applies to stages and the views within any such a stages as well.

The “Undo” button 110 functions to undo one or more previous operations. For example, suppose the sonographer has copied a view into memory and then pasted that view into the wrong location. The sonographer can simply click the “Undo” button 110 to undo the paste operation, select another location and re-paste the view in the correct location. A typical implementation would allow the sonographer to undo multiple previous operations up to some pre-defined limit.

The example of the present invention illustrated in FIG. 4 overcomes the shortcomings of the prior art user interface discussed above. Stages and views such as, for example, stage Rest 112 and LAX A view 114 clearly show their properties at all times on the display. This facilitates ease of comparison and eliminates the need to click between various views and stages in the tree-like structure of the prior art user interface. Likewise, instead of being represented by only textual names, each view is denoted by an image or icon representative of a typical such image previously captured with the ultrasound imaging system. This means of graphically identifying views makes it easier for the sonographer to remember exactly what each view is for and what type of image will result during examination and/or reporting. Lastly, as has been discussed above, this user interface allows for easy configurability or new and existing protocols by allowing for easy movement and organization of existing protocol views. The ability to drag and drop graphical representations of the stages and views is intuitive and efficient and represents an improvement over the prior art interfaces.

What is claimed is:

1. In an ultrasound diagnostic imaging system, a method for managing an ultrasound imaging protocol having at least one protocol stage, the method comprising:
displaying a user interface including graphical representations of ultrasound images characteristic of ultrasound images to be acquired during respective steps of the protocol stage;
manipulating at least one of the displayed graphical representations of ultrasound images;
changing at least one protocol stage in accordance with the graphical representation manipulation to provide a revised ultrasound imaging protocol; and
after providing the revised ultrasound imaging protocol, saving the revised ultrasound imaging protocol in the system.

2. The method of claim 1 wherein managing an ultrasound imaging protocol comprises creating a new ultrasound imaging protocol.

3. The method of claim 2 wherein displaying graphical representations of ultrasound images comprises displaying a pre-existing library of graphical representations of ultrasound images and displaying a stage view of the new protocol configured to receive at least one graphical representation from the library.

4. The method of claim 3 wherein manipulating at least one of the displayed graphical representations of ultrasound images comprises dragging at least one graphical representation of an ultrasound image from the library and dropping the graphical representation into the stage view.

5. The method of claim 4 wherein manipulating at least one of the displayed graphical representations of ultrasound images further comprises creating a new graphical representation of an ultrasound image and storing the new graphical representation in the library prior to dragging the graphical representation from the library and dropping it into the stage view.

6. The method of claim 5, wherein creating a new graphical representation of an ultrasound image comprises reading a stored image from an image file or scanning in a new ultrasound image.

7. The method of claim 3 wherein manipulating at least one the displayed graphical representations of ultrasound images comprises editing the properties of at least one graphical representation of an ultrasound image in the library.

8. The method of claim 1 wherein managing an ultrasound imaging protocol comprises editing a pre-existing ultrasound imaging protocol.

9. The method of claim 8 wherein displaying graphical representations of ultrasound images comprises displaying the graphical representations of the ultrasound images of at least one stage of the protocol being edited.

10. The method of claim 9 wherein manipulating at least one of the displayed graphical representations of ultrasound images comprises re-ordering the displayed graphical representations of ultrasound images by dragging at least one graphical representation and dropping the graphical representation into a new position within the protocol.

11. The method of claim 9 wherein manipulating at least one of the displayed graphical representations of ultrasound images comprises editing the properties of at least one graphical representation of an ultrasound image of the protocol being edited.

12. An ultrasonic diagnostic imaging system comprising:
an ultrasound image display for displaying an ultrasound image; a processor coupled to the ultrasound image display; a user interface coupled to the processor; and
an analysis package stored on a computer readable medium and operatively connected to the processor, the analysis package providing a user the ability to manage an ultrasound imaging protocol, the analysis package being configured to:
display graphical representations of ultrasound images characteristic of ultrasound images to be acquired during respective steps of a protocol stage;
allow manipulation of at least one of the displayed graphical representations of ultrasound images;
change at least one protocol stage in accordance with the graphical representation manipulation to provide a revised ultrasound imaging protocol; and
save the revised ultrasound imaging protocol in the system.

13. The ultrasonic diagnostic imaging system of claim 12, wherein the analysis package comprises hardware and software which is responsive to image data produced by the ultrasonic diagnostic imaging system.

14. The ultrasonic diagnostic imaging system of claim 13, wherein the analysis package can be configured for a particular type of diagnostic application.

15. The ultrasonic diagnostic imaging system of claim 14, wherein the particular type of diagnostic application comprises one of OB, GYN, general imaging, vascular, breast, small parts, or cardiology.

16. The ultrasonic diagnostic imaging system of claim 15, wherein the particular type of diagnostic application comprises OB, and wherein the exam protocol is a fetal exam protocol.

17. The method of claim 12 wherein managing an ultrasound imaging protocol comprises creating a new ultrasound imaging protocol.

18. The method of claim 17 wherein displaying graphical representations of ultrasound images comprises displaying a pre-existing library of graphical representations of ultrasound images and displaying a stage view of the new protocol configured to receive at least one graphical representation from the library.

19. The method of claim 18 wherein manipulating at least one of the displayed graphical representations of ultrasound images comprises dragging at least one graphical representation of an ultrasound image from the library and dropping the graphical representation into the stage view.

20. The method of claim 19 wherein manipulating at least one of the displayed graphical representations of ultrasound images further comprises creating a new graphical representation of an ultrasound image and storing the new graphical representation in the library prior to dragging the graphical representation from the library and dropping it into the stage view.

21. The method of claim 18 wherein manipulating at least one of the displayed graphical representations of ultrasound images comprises editing the properties of at least one graphical representation of an ultrasound image in the library.

22. The method of claim 12 wherein managing an ultrasound imaging protocol comprises editing a pre-existing ultrasound imaging protocol.

23. The method of claim 22 wherein displaying graphical representations of ultrasound images comprises displaying the graphical representations of the ultrasound images of at least one stage of the protocol being edited.

24. The method of claim 23 wherein manipulating at least one of the displayed graphical representations of ultrasound images comprises re-ordering the displayed graphical representations of ultrasound images by dragging at least one graphical representation and dropping the graphical representation into a new position within the protocol.

25. The method of claim 23 wherein manipulating at least one of the displayed graphical representations of ultrasound images comprises editing the properties of at least one graphical representation of an ultrasound image of the protocol being edited.

26. The method of claim 25, wherein editing the properties comprises changing one of the imaging mode, annotation, or automatic launch of calculations or measurements.
